(19) United States
(12) Patent Application Publication
(10) Pub. No.: US 2017/0181666 A1
(43) Pub. Date: Jun. 29, 2017

(54) LIGHT-BASED ENDOLUMINAL SIZING DEVICE

(71) Applicant: Spiration, Inc. d.b.a. Olympus Respiratory America, Redmond, WA (US)

(72) Inventor: Timothy J. Johnson, Kent, WA (US)

(73) Assignee: Spiration, Inc. d.b.a. Olympus Respiratory America, Redmond, WA (US)

(21) Appl. No.: 15/300,443

(22) PCT Filed: Mar. 30, 2015
(86) PCT No.: PCT/US15/23402

§ 371 Date: Sep. 29, 2016

Related U.S. Application Data
(60) Provisional application No. 61/973,105, filed on Mar. 31, 2014.
(60) Provisional application No. 61/973,169, filed on Mar. 31, 2014.
(60) Provisional application No. 61/973,142, filed on Mar. 31, 2014.
(60) Provisional application No. 61/973,137, filed on Mar. 31, 2014.
(60) Provisional application No. 61/973,110, filed on Mar. 31, 2014.

(51) Int. Cl. A61B 5/107; A61B 90/00; A61B 1/267; A61B 1/012; A61B 1/06

(52) U.S. Cl. CPC A61B 5/1079; A61B 1/012; A61B 1/0623; A61B 1/0676; A61B 1/0684; A61B 1/267; A61B 5/1076; A61B 90/06; A61B 1/0661; A61B 1/07

(57) ABSTRACT

Devices and methods for measuring size of airways to lung are disclosed. A light-based endoluminal sizing device can comprise a centering tool, a light source, and a marker. A size of an airway can be approximated or determined based on a distance between the marker and the light source when light from the light source intersects the marker at an interface between the marker and the airway wall. In some cases, the size of the airway can be approximated from observing an intersection between a light pattern and the airway wall.

### REFERENCE TO RELATED APPLICATIONS

Any and all applications for which a foreign or domestic priority claim is identified in the Application Data Sheet as filed with the present application are hereby incorporated by reference under 37 CFR 1.57. This application claims priority to U.S. Provisional Patent Application No. 61/973,137, filed Mar. 31, 2014, titled Light-Based Endoluminal Sizing Device (Atty. Ref. No. SPIRTN.103PR). The entire disclosures of each of the foregoing applications are hereby made part of this specification as if set forth fully herein and incorporated by reference for all purposes, for all that each contain.

### BACKGROUND

Field

This disclosure relates to devices that measure the size of airways to lungs.

Related Art

Balloons filled with fluid are typically used to measure the size of airway to lungs. However, provided herein are devices and methods that can help measure a size of airways to lungs using light beams, for example.

### SUMMARY

The present technology relates to devices and methods for measuring the size of airways to lungs.

A light-based endoluminal sizing device for sizing a body lumen can comprise, for example, a marker deployable in airway lungs, the marker further comprising a central axis, a centering tool that perpendicularly connects an axis of the lumen to the marker, and a light source disposed substantially collinear to the central axis of the marker. The light source can emit light beams radially outward at a known angle. The centering tool can align the light source with an axis of the lumen. Intersecting the light beams emitted from the light source with the marker can indicate a size of the body lumen.

The marker for a light-based endoluminal sizing device can comprise a hub and a plurality of extension members extending radially outward from the hub. A light-based endoluminal sizing device can further comprise, for example, a first configuration that permits the marker to be flexibly disposed within a catheter and be transported through the catheter. A light-based endoluminal sizing device can further comprise, for example, a second configuration that places the marker outside of the catheter such that the plurality of extension members contact the body lumen.

The centering tool can further comprise a centering rod. The centering rod can be configured to telescopically elongate. The first light source can be movable substantially parallel to an axis of a lumen with respect to the marker. The marker can be a second light source movable substantially parallel to an axis of a lumen with respect to the first light source.

A method of using a light-based endoluminal sizing device for sizing a body lumen can comprise, for example, locating a marker on an endoluminal wherein the marker contacts the lumen at a marker contact point, aligning a light pattern to intersect the marker contact point by adjusting a position of the light source relative to the marker, wherein the light pattern emits light from a light source at a known angle relative to a centering tool; and determining an endoluminal size by measuring a distance between the light source and the marker when the light pattern intersects the marker. A method of using a light-based endoluminal sizing device for sizing a body lumen can comprise, for example, aligning the light pattern comprises moving the marker relative to the light source. Aligning the light pattern can comprise, for example, using an electronic sensor configured to communicate alignment of the marker relative to the light source to a user. Aligning the light pattern can comprise, for example, using a bronchoscope. The light pattern can be a conical light beam configured to emit light at a set angle. Determining an endoluminal size can comprise using a visual indicator configured to display a size of the body lumen using the distance measured between the light source and the marker. A method of using a light-based endoluminal sizing device for sizing a body lumen can further comprise, for example, delivering the marker to the body lumen using a catheter, deploying the marker by distally pushing the marker away from the catheter, and retracting the marker by proximally pulling the marker toward the catheter.

A light-based endoluminal sizing device for sizing a body lumen can comprise a marker. The marker can be deployable in airway lungs. The marker can further comprise a central axis. The centering tool can be connected to the marker. The centering tool can be configured to align the marker with an axis of the body lumen. The sizing device can comprise a first light source. The first light source can be disposed substantially collinear to the central axis of the marker. The light source can emit light beams radially outward at a known angle. The centering tool can align the first light source with the axis of the body lumen. Intersecting light beams emitted from the first light source with the marker can indicate a size of the body lumen.

The marker can comprise a hub. The light-based endoluminal sizing device can comprise a plurality of extension members extending radially outward from the hub. The light-based endoluminal sizing device can comprise a first configuration that permits the marker to be disposed within a catheter and be transported through the catheter. The light-based endoluminal sizing device can comprise a second configuration that places the marker outside of the catheter such that the plurality of extension members contact the body lumen.

The centering tool of the light-based endoluminal sizing device can further comprise a centering rod. The centering rod can be configured to telescopically elongate. The first light source can be movable substantially parallel to the axis of the body lumen with respect to the marker. The marker can be a second light source movable substantially parallel to an axis of a lumen with respect to the first light source.

A method of using a light-based endoluminal sizing device for sizing a body lumen can comprise locating a marker on an endoluminal wall. The marker can contact the lumen at a marker contact point. The method can comprise aligning a light pattern to intersect the marker contact point by adjusting a position of the light source relative to the marker. The light pattern can emit light from a light source at a known angle relative to a centering tool. The method can comprise determining an endoluminal size by measuring a distance between the light source and the marker when the light pattern intersects the marker.

Aligning the light pattern can comprise moving the marker relative to the light source. Aligning the light pattern can comprise using an electronic sensor configured to communicate alignment of the marker relative to the light source to a user. Aligning the light pattern can comprise using a bronchoscope. The light pattern can be a conical light beam configured to emit light at a set angle.

Aligning the light pattern can comprise intersecting the conical light beam with the marker at a point where the marker contacts the body lumen. Determining an endoluminal size can comprise using a visual indicator configured to display a size of the body lumen using the distance measured between the light source and the marker.

The method of using a light-based endoluminal sizing device can further comprise delivering the marker to the body lumen using a catheter. The method can further comprise deploying the marker by distally moving the marker away from the catheter. The method can further comprise retracting the marker by proximally pulling the marker toward the catheter. In some embodiments, the marker is a second light projected onto the endoluminal wall.

A system for approximating a size of a body lumen can comprise a marker configured to transition between a stored configuration and a deployed configuration. The marker can be configured to contact a wall of a body lumen when in the deployed configuration within a body lumen. The system can comprise a light source. The light source can be connected to the marker and slidable toward and away from the marker. The light source can be configured to output a beam of light toward the marker.

The system can comprise a centering mechanism. The centering mechanism can be connected to the light source. The centering mechanism can be configured to maintain the light source in an approximate center of a body lumen when the light source is deployed in a body lumen. The light source can be a mirror configured to reflect light from a light emitter toward the marker. The marker can comprise a hub and a plurality of tangs extending radially outward from the hub.

The light source can be positioned proximal of the marker. The light source member can be positioned distal of the marker. The system can comprise an elongate member connected to the light source. The marker can be connected to and coaxial with the elongate member. The marker can be configured to slide along a length of the elongate member.

The system can comprise an elongate member. The elongate member can be connected to the marker. The light source can be connected to and coaxial with the elongate member. The light source can be configured to slide along a length of the elongate member.

### BRIEF DESCRIPTION OF THE DRAWINGS

The following drawings and the associated descriptions are provided to illustrate the present disclosure and do not limit the scope of the claims.

FIG. 1 illustrates an embodiment of a light-based endoluminal sizing device.

FIG. 2 illustrates an embodiment of a marker having a plurality of tangs.

FIG. 3 illustrates another embodiment of a light-based endoluminal sizing device.

FIGS. 4A-4B illustrate another embodiment of a light-based endoluminal sizing device.

FIG. 5 illustrate a sizing device loaded into a bronchoscope inserted into a lung airway.

FIG. 6 illustrates an embodiment of a multi-lumen device which can be used to deliver an endoluminal sizing device to a target section.

These and other features will now be described with reference to the drawings summarized above. The drawings and the associated descriptions are provided to illustrate embodiments and not to limit the scope of any claim. Throughout the drawings, reference numbers may be reused to indicate correspondence between referenced elements.

### DETAILED DESCRIPTION

Although certain preferred embodiments and examples are disclosed herein, inventive subject matter extends beyond the specifically disclosed embodiments to other alternative embodiments and/or uses and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of illustrating various feature combinations and embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

Airways may be measured before devices can be placed into lungs. Measuring airway sizing can be done to reduce the likelihood of a device having an inappropriate size being used in an airway.

In some cases, balloons tilled with fluid arc used to measure airway size. The volume of fluid in the balloon can indicate the expanded diameter of the balloon, and volume of fluid needed to gently expand the balloon against the airway wall can indicate the airway dimensions (e.g. diameter). Some catheters use tangs to indicate when an airway is too big for the device. With flexible tangs of different heights away from the center of a catheter, the operator can determine if the airway is in a certain range of diameters. When the members touch the wall of the airway, the user can read the diameter off of the handle.

In some cases, the airway wall can have varying degrees of compliance and mechanical sizing tools can displace the walls leading to measurement error. Tools that do not self-center can erroneously measure a cross-sectional chord rather than a diameter. Some airways are not cylindrical, but can be tapered along the length or non-circular in cross section.

Disclosed herein are devices that can measure the airways size while contacting the wall with minimal force. The ability to read a scale directly can speed the procedure.

### Sizing Device

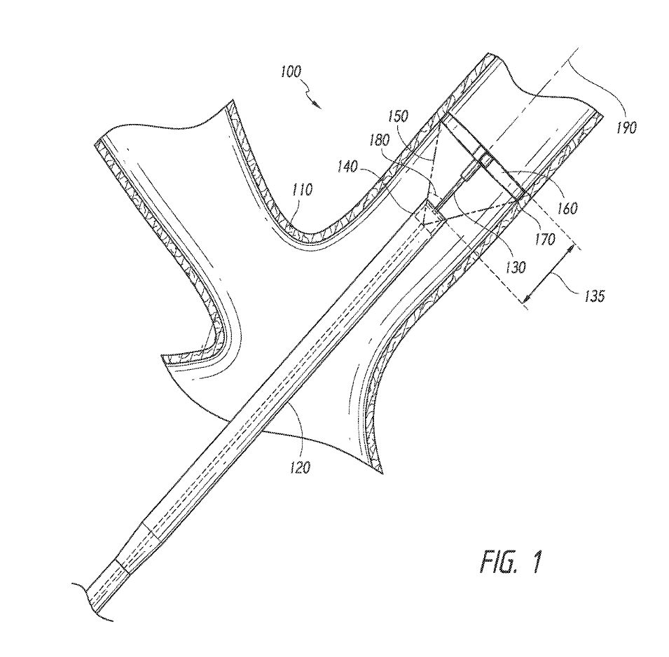

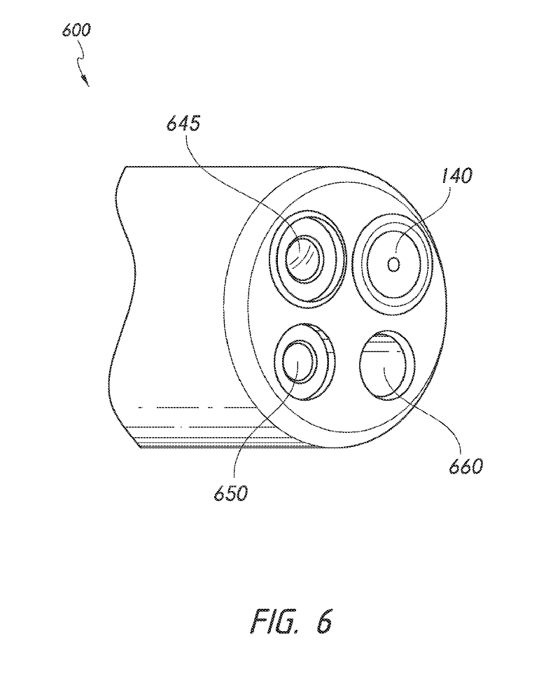

FIG. 1 shows a light-based endoluminal sizing device 100 comprising a light source 140 and a marker 160. The light source 140 can be configured to emit/reflect light (e.g., a beam, spectrum, or other light emission) through a catheter 120. In some embodiments, the light source 140 is a mirror or other device/structure configured to reflect and/or redirect light from a light emitter. The light emitter (not shown) can comprise a lamp, fiber-optic cable, laser, or other light-emitting device configured to emit light through the catheter 120 or other elongate passage. The device 100 can include a light sensor/camera separate from the light source 140. For example, as shown in FIG. 6, the light source 140 can occupy a separate aperture of a multi-lumen bronchoscope 600 from a camera lens 645. The device 100 can include a mirror 140 configured to deflect the light 142 from the light source.

The marker 160 can comprise a reflective material, such as a mirror. The marker 160 can further comprise a centering tool 130. The centering tool 130 can be a separate mechanism detachable and/or independent from the marker 160. The light source 140 can be disposed on or near the distal end of the catheter 120. The light source can be located on or substantially close to an axis 190 of a lumen 110 into which the catheter 120 is deployed. The centering tool 130 can be configured to position the marker 160 relative to the light source 140. For example, the centering tool 130 can align the marker 160 to be substantially collinear to the axis of the lumen 190 and/or to the light source 140.

The light source 140 can be positioned off-axis, separate from the marker 160 and/or the catheter 120. For example, the light source 140 can be a light that emits/reflects from an end of a separate tube. In some embodiments the light source 140 and marker 160 are housed at least partially within the same catheter 120 during and/or after deployment of the device 100.

A light beam 150 can be projected distally at a known angle 180 away from the light source 140. The light beam 150 can be a laser beam, such as a laser beam used to measure a distance between an object using laser triangulation method. The light beam 150 can be projected in a form of a conical beam (e.g. laser light emerging from an optical fiber with known numerical aperture). The light beam 150 can be reflected off of a mirror or other reflective surface. The light beam 150 projected from the light source 140 can contact the lumen 110.

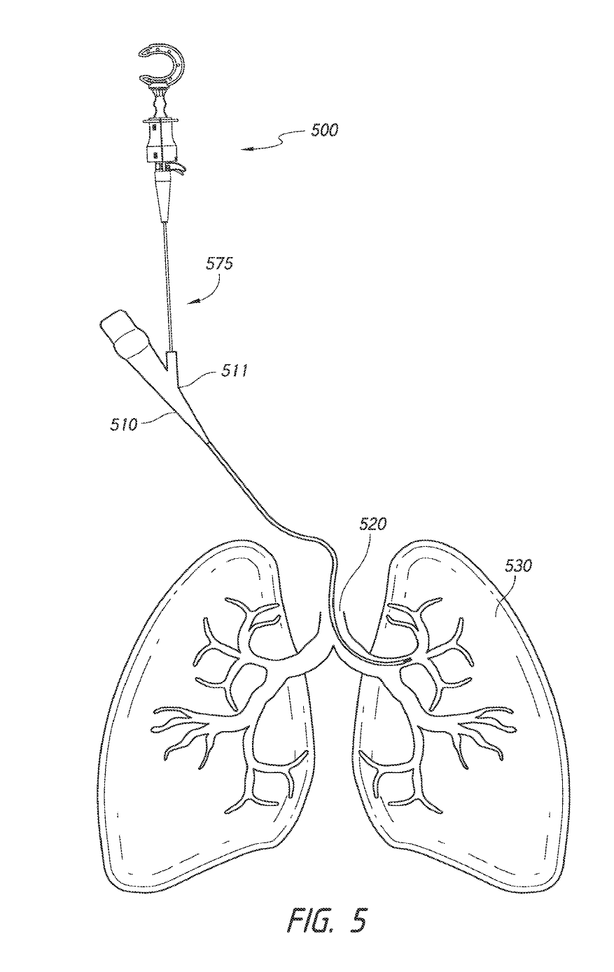

The light-based endoluminal sizing device 100 can be inserted to the body lumen 110 through a delivery device, such as, for example, a bronchoscope 510. FIG. 5 shows an example proximal end 500 of a sizing device loaded into a bronchoscope 510 inserted into a lung airway 520. The marker 160 can be shaped and sized to fit inside a catheter 120, or other tube. A user may place the light-based endoluminal sizing device 100 inside a body lumen using a bronchoscope 510. The user may operate the light-based endoluminal sizing device 100 to deploy and/or expand the marker 160.

In a first configuration, the marker 160 can be folded inside a smaller diameter of an aperture within the delivery device. For example, the marker 160 can be folded to fit inside a working channel 650 of the bronchoscope 600, shown in FIG. 6. In a second configuration, the marker 160 can be pushed out from the working channel 650 to be deployed. In some embodiments, the marker 160 is positioned within the catheter 120 prior to deployment, and the catheter 120 is withdrawn from the marker 160 upon deployment.

Once deployed, a portion of the marker 160 can be placed against the lumen wall 110 at a marker contact point 170. The centering tool 130 can be configured to restrict movement of the marker 160 relative to the light source 140. For example, the centering tool 130 can limit the distance and angle in which the marker 160 can be moved away from the light source 140. A user may retract the marker 160 into the catheter 120 by moving the marker 160 toward the catheter 120 and into the catheter 120. The marker 160 can be pulled by using a pull wire. For example, the marker 160 can be tapered proximally such that the marker 160 folds into the catheter 120 (e.g., or into the channel for instruments 650 when pulled proximally into the channel 650). A separate sheath can be used to wrap the marker 160 so that the marker 160 is retracted and folded to be transported along the catheter 120.

The marker 160 can be placed against the lumen wall 110 at a distance 135 distal to the light source 140. The distance 135 between the light source 140 and the marker 160 can be adjusted. For example, the device 100 can include a proximal handle configured to permit movement (e.g., axial movement) of the light source 140 with respect to the marker 160. The distance 135 can be observed and/or calculated via markings on a proximal end of the device 100 and/or via markings on a distal end (e.g., at or near the centering tool 130) of the device 100.

The centering tool 130 can be used to position the light source 140 relative to the body lumen 110. For example, as shown in FIG. 1, the centering tool 130 can align the light source 140 to be collinear with the axis of the lumen 110. The axis of the light source 140 can be aligned with the axis of the lumen while the light beam 150 intersects the marker 160 at a marker contact point 170. In this configuration, airway size can be determined from the known angle 180 and the longitudinal distance 135 between the light source 140 and the marker 160.

The intersection of the light beam 150 and the marker 160 can be visually confirmed via a medical imaging device (e.g., an endoscopic or bronchoscope). The marker 160 can comprise a reflective material, such that the marker 160 can reflect light projected distally from the light source 140. The user may use the reflection from the marker 160 to indicate where (e.g., along a radial portion of the marker 160) the light pattern intersects the marker. The reflection from the marker 160 can be detected or sensed electronically to determine where the light pattern intersects the marker 160.

The camera lens 645 can further comprise reticles or crosshairs. The reticles can indicate the dimension of the lumen. For example, the user can determine endoluminal dimension by viewing an intersection of the marker contact point 170 and the reticle when the marker 160 is placed at a set distance 135 away from the camera lens 645.

### Centering Tool

The centering tool 130 can be separate from the marker 160. In some embodiments, the centering tool 130 is connected to and/or integral with the marker 160. The centering tool 130 can be placed on a catheter 120. In some embodiments, the centering tool 130 is configured to fit within and extend from a catheter 120. The centering tool 130 can have different forms. For example, a centering tool can be a balloon or a mechanical device. In some embodiments, the centering tool 130 is a rod, tube, wire, fiber, or other structure connected to the marker.

The marker 160 and the centering tool 130 can be combined into a single component. For example, the marker 160 can comprise a mechanical centering tool, and the distance (e.g., axial distance) between the centering tool position and the light source 140 can determine sizing of the lumen 110. This combination component can be attached to the end of a rod, tube or a wire. In some embodiments, the light source 140 can slide along the centering tool rod, such that the distance 135 between the light source and the marker can be adjusted.

### Marker

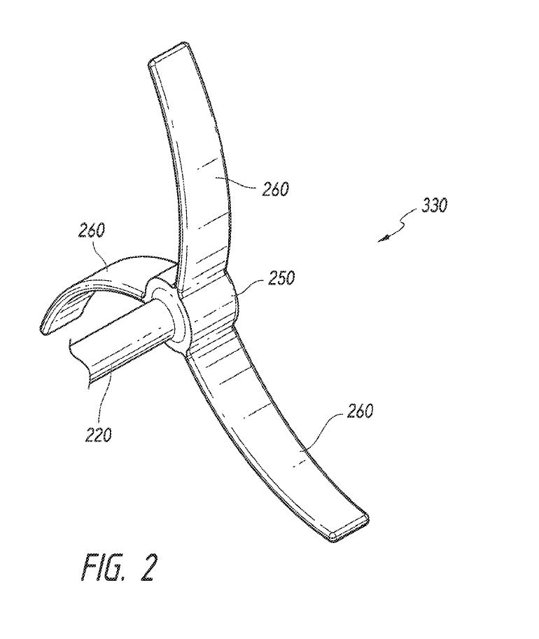

FIG. 2 shows a sample marker 330 having a plurality of tangs 260. The marker having plurality of tangs 330 can comprise a mechanical apparatus. For example, the marker 330 can comprise a centering tool 220 and a plurality of tangs 260. The centering tool 220 can further comprise a hub 250. The plurality of tangs 260 can extend radially outward from the hub 250 of the centering tool 220. The plurality of tangs 260 can connect to the centering tool 220 at the hub 250. The plurality of tangs 260 can comprise any mechanical structure that can be folded into an aperture of a medical delivery device. In some embodiments, the tangs can be configured to fit inside a channel for instruments 650 of a multi-lumen bronchoscope 600 shown in FIG. 6. In some embodiments, the tangs 260 can be folded into the catheter 120. The plurality of tangs 260 can comprise a flexible member, such as a spring or a spiral. The plurality of tangs 260 can be petals of metallic foils, thin sheets of metal made of a shape-memory material (e.g. Nitinol), umbrella ribs, and/or other suitable structures/materials. In some embodiments, the marker 330 comprises a plurality of coaxial tube portions (not shown), each portion forming at least one of the tangs 260. The coaxial tube portions can be laser cut. In some embodiments, the coaxial tube portions are laser welded to each other. One or more of the tangs 260 can include a twist (e.g., a helical pattern) at or near the hub 250. The twist can be configured to facilitate bending of the tangs 260 in a distal direction (e.g., away from the centering tool 220) when the marker 330 is retracted into a catheter or other lumen.

The plurality of tangs 260 can be deployed when the user distally pushes the marker having plurality of tangs 330 out and away from a catheter 120. In some embodiments, the plurality of tangs 260 are deployed when a user withdraws the catheter 120 from the marker 330 at a deployment site within the lumen 110. Each of the plurality of tangs 260 can have a size and elasticity configured to expand to the size (e.g. circumference) of the lumen wall. For example, once deployed, the plurality of tangs 260 can be expanded radially to come in contact with the lumen wall 110. The plurality of tangs 260 can be configured to exert sufficient force against the lumen wall 110 to inhibit or prevent inadvertent movement of the marker 330 within the lumen 110 (e.g., so that a user may accurately measure the size of the lumen/airway). The force exerted against the lumen wall 110 by the plurality of tangs 260 can be such that damage to the lumen wall 110 due to movement of the plurality of tangs 260 (e.g. expansion, contraction, and longitudinal movement) is minimized. The shape of the plurality of tangs 260 can be configured to minimize trauma abrasion against the lumen wall. For example, the plurality of tangs 260 can have a blunt tip. The tip of the plurality of tangs 260 can be configured not to come in contact with the lumen wall, such as by having the tip point toward a central axis of the plurality of tangs 260, for example.

The plurality of tangs 260 can be folded into a tube by being pulled proximally into a tube or a catheter 120 of a bronchoscope, For example, the plurality of tangs 260 can be tapered proximally, such that when a user pulls the marker having plurality of tangs 330 into the tube or the catheter, the plurality of tangs 260 are spirally folded from being tucked inside of the tube.

One or more tangs can be used. For example, a marker having plurality of tangs 330 can comprise four or more tangs 260. The plurality of tangs 260 can be webbed. For example, a marker having plurality of tangs 330 can comprise a plurality of tangs 260 configured to expand a fabric or a sheet made of polymer. Such configuration can be used, for example, to minimize the abrasion to the surface of the lumen wall 110.

Sizing Device with Distal Light Source

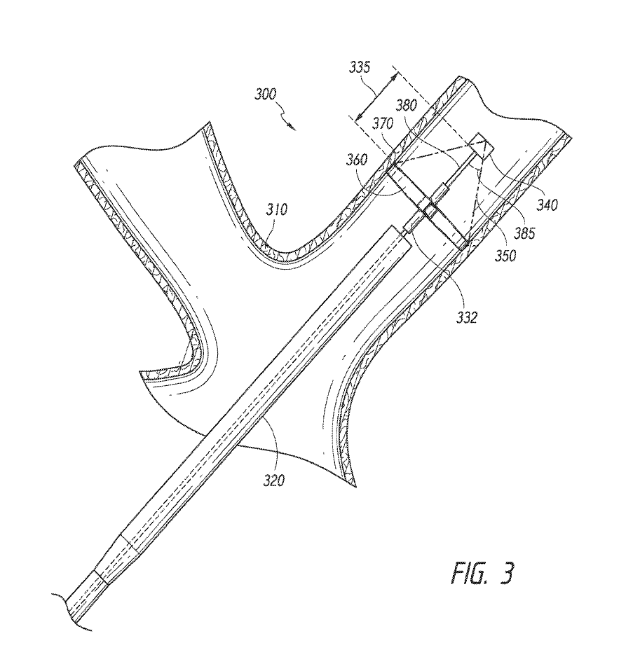

FIG. 3 illustrates a sample light-based endoluminal sizing device 300 having a distal light source. The light-based endoluminal sizing device 300 can comprise a marker 360, a light source 340, and a centering tool 380. The centering tool 380 can comprise a centering tool hub 332. In some embodiments, the centering tool 380 comprises a rod or other rigid structure connected to the mirror 340 and/or to the marker 360. The centering tool 380 can further comprise a distal stopper, not shown. For example, the distal stopper can be used to retract the marker 360 and the light source 340 into the catheter 320. The light source 340 may be configured to serve as a stopper to retract the marker 360 to the catheter 320.

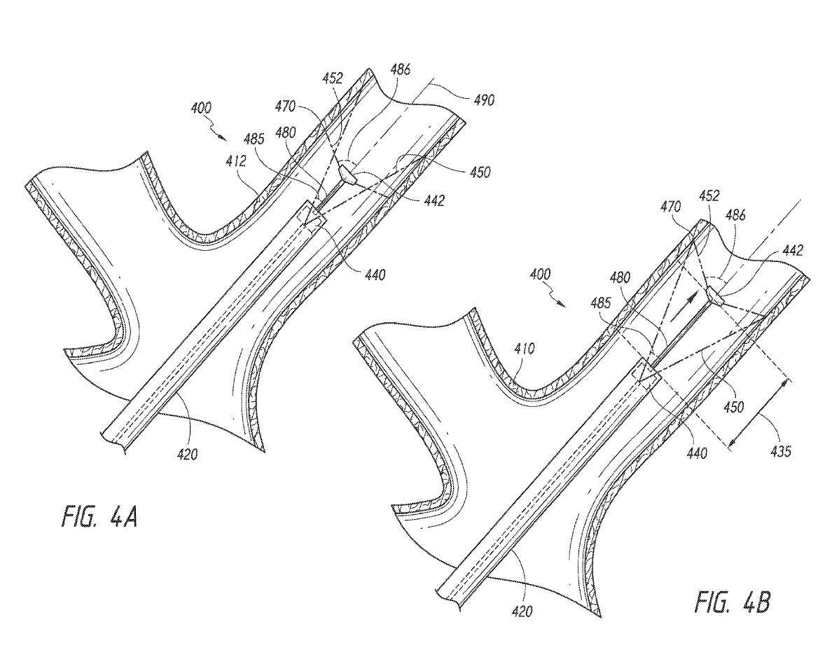

The marker 360 can be a mechanical marker, such as a marker shown and described in reference to FIG. 2. The marker 360 can contact the lumen 310 at a marker contact point 370. The marker 360 can comprise a sensor configured to determine the location of the marker contact point 370. The marker 360 can be a second beam, such as beam from a proximal mirror in the two-mirror configuration of an endoluminal sizing device shown and described in reference to FIG. 4. The marker 360 can be a balloon or an umbrella.

As illustrated in FIG. 3, the marker 360 can be placed proximally to the light source 340, such that a light beam 350 can be emitted toward the proximally placed marker 360. The centering tool 380 can connect the marker 360 to a catheter 320 or a tube connected to a medical delivery device (e.g., a bronchoscope or other endoscope). The centering tool 380 can be configured to position the light source 340 to be substantially collinear with the lumen 310. For example, the centering tool 380 can be configured to position the light source 340 to be collinear to the hub 332 of the centering tool 380 and/or to the marker 360. The centering tool 380 can extend distally and away from the marker 360. The light source 340 can be connected to the distal end of the centering tool 380. The light source 340 can be configured to emit/reflect light proximally towards the marker at a known angle 385. For example, a light source 340 comprising a mirror can receive light emitted distally from a medical delivery device and reflect the light proximally.

A user may deploy the marker 360 by pushing the marker 360 away from the catheter 320. In some embodiments, the marker 360 is deployed by withdrawing the catheter 320 from the marker 360 when the marker 360 is positioned at the site of interest. Once the marker 360 is deployed to rest against the lumen wall 310, the distal light source 340 can be pushed/pulled independently from the marker 360 and toward/further away from the marker 360. When the light source 340 is at a certain distance 335 away from the marker 360, the light beam 350 can intersect the marker 360 at a marker contact point 370. When the light beam 350 intersects the marker 360, a user may determine the size of the airway by knowing the distance 335 that the light source 340 is placed relative to the marker 360, and the pre-set angle 385. The user may determine the size of airway without intersecting the light beam 350 at the marker contact point 370. For example, the light beam 350 may intersect the marker 360 at a certain radius away from the marker hub. In such settings, the user may determine the size of the airway by knowing the radius where the light beam 350 intersects the marker 360. For example, the marker 360 may include one or more markings to indicate a radial distance at varying points along the radius of the marker 360 from the airway wall.

A user may visually determine when the light beam 350 intersects the marker 360. For example, the user may determine the intersection with the marker contact point 370 by using a bronchoscope camera. The endoluminal sizing device 300 can further comprise a sensor used to determine when the light beam 350 intersects the marker 360 at the marker contact point 370.

The light source 340 can be positioned off-axis. For example, the light source 340 can be positioned to contact the lumen wall 310. The beam 350 can be a conical beam with an angle 385, with an axis parallel to the lumen axis. The conical beam 350 projected to the lumen 310 may form an angled ellipse in which the most distal point marks a position of the substantially longest chord across the lumen from the light source; for example, across the diameter. This most distal point of the projected ellipse can be used to measure a diameter. The most distal point of the projected beam or a discontinuity in the beam can be used to measure a specified chord across the lumen. The light source can be configured to adjust the known angle 385. A user may determine the size of the lumen by adjusting the known angle 385 so that the beam intersects the marker at the marker contact point 370.

Sizing Device with Multiple Light Sources

FIGS. 4A-4B illustrate an example shape of a light-based endoluminal sizing device 400 having two light sources. The light-based endoluminal sizing device 400 can comprise, for example, a catheter 420, a centering tool 480, a proximal light source 440, and a distal light source 442. The centering tool 480 can be configured to allow movement of the distal light source 442 substantially collinear to an axis of the lumen 490.

The proximal light source 440 can be configured to emit/reflect a first light beam 450 at a first angle 485. The distal light source 442 can be configured to emit/reflect a second light beam 452 at a second angle 486. The second angle 486 can be greater than the first angle 485. The proximal light source 440 can be affixed to a distal end of the catheter 420. The first and second angles 485 can form an angle less than 90 degrees relative to the centering tool 480. In some embodiments, the second angle 486 is greater than 90 degrees.

A user may operate the light-based endoluminal sizing device having two light sources 400 by placing the catheter 420 having the light system 400 in the lumen 410. The proximal light source 440 and the distal light source 442 can each comprise mirrors configured to reflect light. The catheter 420 can house the light-based endoluminal sizing device having two light sources 400 as the device is transported inside the catheter 420. A light source can be switched on or off during operation of the device to project the first and second light beams 450, 452. For example, a lamp, such as a lamp 640 shown and described in reference to FIG. 6 can be used to project light towards the proximal light source 440 and the distal light source 442. In some embodiments, a light source (e.g., fiber optic, laser, or other light source) can emit light through the catheter 420 to reflect off of mirrors 440, 442. In some embodiments, the proximal light source 440 and the distal light source 442 can each be separate sources of light where lights originate from each light source.

As shown in FIG. 4B, the distance 435 between first proximal light source 440 and the distal light source 442 can be used to determine the airway size. The first and second beams 450, 452 can be annular rings (e.g., from a diffraction pattern) or line(s). The distance 335 between the first proximal light source 440 and the distal light source 442 can be measured with a scale (e.g. a needle that moves with the position of the light source next to an airway size scale or a calibrated distance) or a linear encoder on the proximal end of the device.

At a first position, shown in FIG. 4A, the distal light source 442 is released from the catheter 420. A user may operate the device 400 to move the distal light source 442 in a distal direction to a second position, shown in FIG. 4B. Each of the first and second light beams 452, 450 can contact the lumen 410. The second light beam 452 can intersect the first light beam 450 at a beam contact point 470 when the distal light source 442 is at a certain distance 435 away from the proximal light source 440. The beam contact point 470 can be a point where the first and second light beams 450, 452 intersect each other and the lumen wall. The user may determine the size of the lumen 410 by knowing the first and second angles 485, 486 and the distance 435 between the proximal light source 440 and the distal light source 442 at the second position. The user may visually determine when the first and second light beams 452, 450 intersect by looking through a bronchoscope. The light-based endoluminal sizing device having two light sources 400 can comprise one or more sensors that can inform the user when the first and second light beams 452, 450 intersect at a point of the lumen 410.

The proximal light source 440 can be movable relative to the catheter 420. The first and second light beams 452, 450 can be reflected proximally, such that each of the first and second angles 485, 486 form angles greater than 90 degrees relative to the centering tool 480. Either or both of the first light source 440 and/or the second light source 442 can be moved and adjusted. The distance 435 can depend on the sizing of the bronchial lumen 410. The first and second light beams 452, 450 can be of different colors.

### Light Source

The light sources 140, 340, 440 can be configured to emit and/or reflect light. For example, the light sources 140, 340, 440 can be a lamp, such as an LED lamp, fiber optic light source, or a laser. The light sources 140, 340, 440 can be a reflective material, such as a mirror. The light source 340 can be a transmissive optical device, such as a lens. The light sources 140, 340, 440 can be a combination of one of more of aforementioned optical devices, such as an LED lamp coupled with a mirror. The light sources 140, 340, 440 can be a beam pointed off-axis, a conical beam projected from a lens, a reflected laser beam, or a projection of a laser diffraction pattern (e.g. a line, an annular ring, a zig-zag crown, or another shape). The light source can be pointed off-axis by reflection (e.g. a mirror), refraction (e.g. a lens), diffraction (e.g. a grating or hologram), or emergence from optical fibers.

The light source can comprise a camera. For example, the light source can comprise a camera attached to a bronchoscope. The camera can further comprise markers. For example, the markers can comprise a plurality of concentric circles inscribed a camera lens, or any other type of visual indicator on a lens. A user may determine the distance between the marker 360 and the camera by aligning the marker-beam intersection 370 with the plurality of concentric circles or crosshairs.

The light source can be a beam pointed off-axis, a conical beam projected from a lens, a reflected laser beam, or a projection of a laser diffraction pattern (e.g. a line, an annular ring, a zig-zag crown, or another shape). The light source can be pointed off-axis by reflection (e.g. a mirror), refraction (e.g. a lens), diffraction (e.g. a grating or hologram or emergence from optical fibers.

### Multi-Lumen Catheter

FIG. 6 shows a distal end of a multi-lumen bronchoscope 600 which can be used to deliver an endoluminal sizing device to a target section. The multi-lumen bronchoscope 600 can comprise a plurality of apertures, where each aperture can be in communication with a channel and/or device used for various different purposes. For example, the multi-lumen catheter 600 can comprise a light source or a lamp 640, a camera lens 645, a working channel 650, and/or an air and fluid port 660.

The working channel 511, 650 can be used to transport the endoluminal sizing device. For example, as shown in FIG. 5, device 500 can be inserted into a proximal end of a bronchoscope 510 to access a working channel 5. The endoluminal sizing device 500 can be inserted through the working channel 511 and be transported into a lung airway 520.

### Terminologies/Additional Embodiments

Various modifications to the implementations described in this disclosure may be readily apparent to those skilled in the art, and the generic principles defined herein may be applied to other implementations without departing from the spirit or scope of this disclosure. Thus, the claims are not intended to be limited to the implementations shown herein, but are to be accorded the widest scope consistent with this disclosure, the principles and the novel features disclosed herein. Additionally, a person having ordinary skill in the art will readily appreciate, the terms “upper” and “lower” are sometimes used for ease of describing the figures, and indicate relative positions corresponding to the orientation of the figure on a properly oriented page, and may not reflect the proper orientation of the device as implemented.

Certain features that are described in this specification in the context of separate implementations also can be implemented in combination in a single implementation. Conversely, various features that are described in the context of a single implementation also can be implemented in multiple implementations separately or in any suitable sub combination. Moreover, although features may be described above as acting in certain combinations and even initially claimed as such, one or more features from a claimed combination can in some cases be excised from the combination, and the claimed combination may be directed to a sub combination or variation of a sub combination.

Similarly, while operations are depicted in the drawings in a particular order, this should not be understood as requiring that such operations be performed in the particular order shown or in sequential order, or that all illustrated operations be performed, to achieve desirable results. Further, the drawings may schematically depict one more example processes in the form of a flow diagram. However, other operations that are not depicted can be incorporated in the example processes that are schematically illustrated. Additionally, other implementations are within the scope of the following claims. In some cases, the actions recited in the claims can be performed in a different order and still achieve desirable results.

In describing the present technology, the following terminology may have been used: The singular forms “a,” “an,” and “the” include plural referents unless the context clearly dictates otherwise. Thus, for example, reference to an item includes reference to one or more items. The term “ones” refers to one, two, or more, and generally applies to the selection of some or all of a quantity. The term “plurality” refers to two or more of an item. The term “about” means quantities, dimensions, sizes, formulations, parameters, shapes and other characteristics need not be exact, but may be approximated and/or larger or smaller, as desired, reflecting acceptable tolerances, conversion factors, rounding off, measurement error and the like and other factors known to those of skill in the art. The term “substantially” means that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide. Numerical data may be expressed or presented herein in a range format. It is to be understood that such a range format is used merely for convenience and brevity and thus should be interpreted flexibly to include not only the numerical values explicitly recited as the limits of the range, but also interpreted to include all of the individual numerical values or sub-ranges encompassed within that range as if each numerical value and sub-range is explicitly recited. As an illustration, a numerical range of “about 1 to 5” should be interpreted to include not only the explicitly recited values of about 1 to about 5, but also include individual values and sub-ranges within the indicated range. Thus, included in this numerical range are individual values such as 2, 3 and 4 and sub-ranges such as 1-3, 2-4 and 3-5, etc. This same principle applies to ranges reciting only one numerical value (e.g., “greater than about 1”) and should apply regardless of the breadth of the range or the characteristics being described. A plurality of items may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. Furthermore, where the terms “and” and “or” are used in conjunction with a list of items, they are to be interpreted broadly, in that any one or more of the listed items may be used alone or in combination with other listed items. The term “alternatively” refers to selection of one of two or more alternatives, and is not intended to limit the selection to only those listed alternatives or to only one of the listed alternatives at a time, unless the context clearly indicates otherwise.

It should be noted that various changes and modifications to the presently preferred embodiments described herein will be apparent to those skilled in the art. Such changes and modifications may be made without departing from the spirit and scope of the invention and without diminishing its attendant advantages. For instance, various components may be repositioned as desired. It is therefore intended that such changes and modifications be included within the scope of the invention. Moreover, not all of the features, aspects and advantages are necessarily required to practice the present invention. Accordingly, the scope of the present invention is intended to be defined only by the claims that follow.

Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment. Conjunctions, such as “and,” “or” are used interchangeably and are intended to encompass any one element, combination, or entirety of elements to which the conjunction refers.

What is claimed is:

1. A light-based endoluminal sizing device for sizing a body lumen comprising:
a marker deployable in airway lungs, the marker further comprising a central axis;
a centering tool connected to the marker and configured to align the marker with an axis of the body lumen; and
a first light source disposed substantially collinear to the central axis of the marker,
wherein the light source emits light beams radially outward at a known angle,
wherein the centering tool aligns the first light source with the axis of the body lumen; and,
wherein intersecting the light beams emitted from the first light source with the marker indicates a size of the body lumen.

2. The device of claim 1, wherein the marker comprises a hub and a plurality of extension members extending radially outward from the hub.

3. The device of claim 2, wherein the device comprises a first configuration that permits the marker to be disposed within a catheter and be transported through the catheter and a second configuration that places the marker outside of the catheter such that the plurality of extension members contact the body lumen.

4. The device of claim 1, wherein the centering tool further comprises a centering rod configured to telescopically elongate.

5. The device of claim 1, wherein the first light source is movable substantially parallel to the axis of the body lumen with respect to the marker.

6. The device of claim I, wherein the marker is a second light source movable substantially parallel to an axis of a lumen with respect to the first light source.

7. A method of using a light-based endoluminal sizing device for sizing a body lumen comprising:
locating a marker on an endoluminal wall, wherein the marker contacts the lumen at a marker contact point;
aligning a light pattern to intersect the marker contact. point by adjusting a position of the light source relative to the marker, wherein the light pattern emits light from a light source at a known angle relative to a centering tool; and
determining an endoluminal size by measuring a distance between the light source and the marker when the light pattern intersects the marker.

8. The method of claim 7, wherein aligning the light pattern comprises moving the marker relative to the light source.

9. The method of claim 7, wherein aligning the light pattern comprises using an electronic sensor configured to communicate alignment of the marker relative to the light source to a user.

10. The method of claim 7, wherein aligning the light pattern comprises using a bronchoscope.

11. The method of claim 7, wherein the light pattern is a conical light beam configured to emit light at a set angle.

12. The method of claim 11, wherein aligning the light pattern further comprises intersecting the conical light beam with the marker at a point where the marker contacts the body lumen.

13. The method of claim 7, wherein determining an endoluminal size comprises using a visual indicator configured to display a size of the body lumen using the distance measured between the light source and the marker.

14. The method of claim 7, further comprising:
delivering the marker to the body lumen using a catheter;
deploying the marker by distally moving the marker away from the catheter; and
retracting the marker by proximally pulling the marker toward the catheter.

15. A system for approximating a size of a body lumen, the system comprising:
a marker configured to transition between a stored configuration and a deployed configuration, the marker configured to contact a wall of a body lumen when in the deployed configuration within a body lumen; and
a light source connected to the marker and slidable toward and away from the marker, the light source configured to output a beam of light toward the marker.

16. The system of claim 15, comprising a centering mechanism connected to the light source and configured to maintain the light source in an approximate center of a body lumen when the light source is deployed in a body lumen.

17. The system of claim 15, wherein the light source is a mirror configured to reflect light from a light emitter toward the marker.

18. The system of claim 15, wherein the marker comprises a hub and a plurality of tangs extending radially outward from the hub

19. The system of claim 15, wherein the light source is positioned proximal of the marker.

20. The system of claim 15, wherein the light source member is positioned distal of the marker.

21. The system of claim 15, comprising an elongate member connected to the light source, the marker connected to and coaxial with the elongate member and configured to slide along a length of the elongate member.

22. The system of claim 15, comprising an elongate member connected to the marker, the light source connected to and coaxial with the elongate member and configured to slide along a length of the elongate member.

23. The method of claim 7, wherein the marker is a second light projected onto the endoluminal wall.
